(19) United States
(12) Patent Application Publication
(10) Pub. No.: US 2007/0270898 A1
(43) Pub. Date: Nov. 22, 2007

(54) Surgical Cannula

(76) Inventor: Kevin O. Lillehei, Denver, CO (US)

Correspondence Address: KOHN & ASSOCIATES, PLLC, 30500 NORTHWESTERN HWY, STE 410, FARMINGTON HILLS, MI, 48334, US

(21) Appl. No.: 10/561,327

(22) PCT Filed: May 26, 2004
(86) PCT No.: PCT/US04/16462

§ 371 Date: Feb. 7, 2007

Related U.S. Application Data
(60) Provisional application No. 60/480,135, filed on Jun. 20, 2003.

(51) Int. Cl. A61B 17/34

(52) U.S. Cl. 606/198

(57) ABSTRACT

There is provided a floating surgical cannula. A method of forming a surgical cannula by inserting a floating surgical cannula at a location in need of surgery is provided.

### BACKGROUND OF THE INVENTION

1. Field Of The Invention

The present invention generally relates to a system and method for the retraction of tissues and insertion of instruments. More specifically, the present invention relates to a cannula for use in the retraction of tissues.

2. Description Of The Related Art

Surgery involves a complex set of manual tasks with numerous limitations, such as a surgeon's vision and manual dexterity. Enhanced vision allows a surgeon to have a clear visual comprehension of the surgical field and manual dexterity includes a surgeon's ability to manipulate surgical instruments without unnecessary restrictions. Despite a movement toward minimally invasive surgery to reduce post-operative side effects and improved cosmetic results, many current surgical techniques are not minimally invasive due to tradeoffs in visualization, illumination, and dexterity within the surgical field.

For many years most surgery was performed using an open field technique. The surgeon made an incision dictated by the need to directly observe the area or field of interest and to insert his or her hand or hands, and/or one or more instruments therein to perform manipulations within the body cavity accessed through the incision. Retractors and assistants help to provide means of access. For many procedures these incisions are as long as 20 centimeters, traumatic, and painful. This translates into a painful recovery, prolonged hospitalization with a slow return to a normal functional state, and significant cost.

An alternative to open surgery, endoscopic surgery, has also been available for many years, though not as widely applied. Through an endoscope, a tubular optical system for viewing the interior of an organ or body cavity, tissues can be observed. An endoscope is used by making a small incision in the appropriate body covering. A hollow tube, or port, usually 10-25 cm in length and 5-30 mm in diameter, is placed through the incision and the endoscope is placed through the hollow tube. Through various other incisions and ports, other instruments can be placed into a body space for manipulation, grasping, cutting, coagulation etc., similar to open surgery. In the abdomen and pelvis, the optical tube is called a laparoscope and the method is referred to as laparoscopic surgery. Endoscopic surgery provides the visualization and illumination of open surgery without the large incisions.

Laparoscopic surgery usually includes a step of expanding the body cavity with air, inducing a state of pneumoperitoneum, which enhances the surgeon's view and ability to make manipulations. This is accomplished by one of two techniques, air insufflation or abdominal wall lifting. Abdominal wall lifting creates negative pressure within the cavity in relation to the atmosphere, drawing in air through a small incision when the wall is lifted. The disadvantage with this technique is that observation is imperfect. A tent is created with a central peak and a collapsed perimeter. Though most-structures have midline attachments, most endoscopic manipulations take place in the periphery. This is where visualization with this technique is worst. Insufflation is a positive pressure system using a medicinal vapor such as carbon dioxide or nitrogen injected into the peritoneal cavity to balloon the abdominal wall. Expansion is more uniform; vision is better. This is the most widely used technique. Because of the positive pressure, however, the abdomen must be sealed to maintain expansion. This requires that all incisions and ports be sealed. Insufflation also has adverse respiratory and hemodynamic consequences due to positive pressure inhibiting chest expansion and venous blood return to the heart.

Though endoscopic surgery has been available for many years, its application has recently increased due primarily to the development of video monitoring equipment. This has allowed all members of the surgical team to observe, though indirectly, what only the surgeon could previously observe through a laparoscope. In some cases visualization is better than with direct observation. This has led to renewed interest and investigation of these techniques.

The benefit of endoscopic surgery is the limited incisional trauma, improved cosmesis, and decreased pain. For several simple techniques, such as laparoscopic cholecystectomy, this has translated into decreased hospitalization and earlier return to normal function, though cost savings is debated.

While some open surgical procedures have been adapted to laparoscopic technique, there are limitations with this method, particularly with more complex procedures. Fundamental problems relate to the access tubes used for inserting the various manipulative instruments. While limiting incisional trauma, the small diameter of these tubes dictates and limits the design of the inserted instruments. To achieve similar function as in open surgery, equipment becomes complex and therefore more expensive. There is also added risk with each access tube. Each tube requires a stab-wound of the body wall, risking injury to contained viscera with each puncture.

Equally important has been the impact on the surgeon's ability to manipulate tissue. While the visual field may have been improved, instruments that insulate the surgeon from the operative field have markedly reduced tactile sensation, depth perception, and proprioceptive awareness of tissues. As the surgeon continually confirms that that which is done is that which is desired, procedural and anesthesia time increase. Furthermore, the limited access enabled by each port dictates that multiple ports be used. As procedural complexity increases, the surgeon must adapt to a continuously changing and less predictable environment than with simple procedures. The number of ports, and the risk and incidence of complications increases. The requirement for highly skilled and coordinated surgical teams also increases. This has resulted in long learning curves and has limited wide application of these procedures for complex cases.

There has been concern about wound contamination during laparoscopic surgery, particularly the implantation of tumor cells. The etiology of this problem is unclear. It may be a systematic problem with a particular element of the technique, such as insufflation where positive pressure venting through the incision results in contamination. Another systematic problem might be direct contamination during specimen removal. The anecdotal occurrence of these problems suggests a more isolated and less systematic error, such as poor tissue handling technique. However, these concerns and the lack of understanding have limited the application of the technique. It would therefore be useful to develop a surgical instrument that enables endoscopy to be utilized in more delicate surgeries. More specifically, it would be useful to develop an instrument that enables endoscopy to be used without concern for contamination of the tissue wherein the surgery is being performed.

Current intracranial endoscopic surgery, however, still significantly limits a surgeon's ability to perform manual tasks once proper visualization and illumination are achieved.

Conventional retractor systems used in surgery include self-retaining blade retractors (e.g. Greenberg, Budde, Sugita, etc.), handheld brain ribbons, endoscopy sheaths, and microendoscopic discectomy retractors. Numerous limitations of these systems exist. For example, the self-retaining blade retractors cause -excessive brain retraction, allow bleeding into the field (rundown), and brain swelling, as well as limited visualization and illumination. The handheld brain ribbon, in addition to having the above disadvantages, has the obvious disadvantage of needing to be handheld, thereby reducing a surgeon's manual dexterity. Endoscopic instruments have limited utility, particularly when they are used in neurosurgical procedures. Currently available designs limit the working channel to several millimeters. This limits the surgeon to performing surgery with probe-like instruments, and thus, current neuroendoscopic intracranial neurosurgery techniques are limited to procedures with holes or fenestrations.

Furthermore, a microendoscopic discectomy retractor can only be used for spine surgery and its dilators are not suited for other types of surgeries, such as brain surgery, because it can damage brain tissues.

In order to overcome some of the problems disclosed above, the prior art discloses an expandable surgical cannula c which can be inserted into a patient in an initially folded up state and then subsequently unfolded to assume a larger diameter to provide an enlarged access portal for the insertion of surgical instruments therethrough subsequent to cannula insertion into a patient's body. Preferably, the diameter of the cannula is increased in size by an obturator passing therethrough.

In a preferred aspect, the expandable cannula includes a plurality of generally planar longitudinally extending wall sections which are pivotally joined together along their lengths to enclose a cannulated passageway, with at least some of the wall sections being foldable one over another such that the cannula has a first cross sectional area when the wall sections are folded one over another and such that the cannula has a second cross sectional area when the wall sections are unfolded, wherein the first cross sectional area is smaller than the second cross sectional area. In optional preferred aspects, systems are provided for locking the cannula into an open unfolded state such that the large diameter passageway therethrough is kept open during surgery. However, the joints can be problematic during use. The fact that the device requires an obturator in order to maintain the cannula in an open position obviates the desire to develop a device that is easy to use.

Additionally, problems can occur during use. Such problems are typically use-dependent. For example, gynecological laparoscopic surgery requires a pelvic assistant to hold and manipulate uterine cannulas at the command of the gynecologist. This complicates the use of the cannula in gynecological surgeries or procedures.

The use of the pelvic assistant during gynecological laparoscopy has several drawbacks. For long procedures, for example sometimes up to 20 minutes in a set position, the assistant becomes physically fatigued and is unable to maintain the desired orientation of the cannula. A further disadvantage is that because the assistant relies on oral instructions from the doctor the correct positioning of the cannula is an iterative process and therefore the time taken to correctly position the cannula is significantly greater than would be the case if the doctor could adjust the position. Further, the use of an assistant increases costs of the procedure, as the assistant becomes a dedicated member of the surgical team.

Cervical dilation is an integral step in many gynecological procedures, including dilation and curettage (D&C) and hysteroscopy. Current methods of dilating the cervix have existed for centuries. These include slow dilation (over the course of hours) with laminaria tents (a dry sponge that expands as it absorbs fluids from the cervix and vagina) or fast dilation (over the course of minutes) with a set of steel sounds/rods of increasing diameter. Serial dilation of the cervix is fraught with risk, including perforation of the uterus and injury to the muscles of the cervix Laminaria tents are unreliable and frequently exhibit “dumbbelling,” where the two ends expand but the portion in the cervix stays contracted. Therefore, between 8-24 larminaria tents may be required for each procedure.

More recently there has been a trend towards total laparoscopic hysterectomy and laparoscopic pelvic floor repair. Various devices have been incorporated to help facilitate this procedure, from uterine manipulators to vaginal tubes. The use of the vaginal tubes has been to provide cervical-vaginal delineation. Some of the uterine manipulators, although efficient, are difficult to use, have various parts that are easily misplaced and are, in general, bulky instruments. Moreover, almost all of them require a pelvic assistant, usually a doctor or a nurse, to hold the manipulator in place during the operation or to move it on command by the laparoscopic surgeon.

In the current art form, during total laparoscopic vaginal delivery, the vaginal vault is divided from the cervix to enable delivery of the uterus through the vagina. Various vaginal tubes have been used to delineate tissue plane and facilitate this procedure. The vaginal tubes may be made from various materials. A metal tube is used by surgeons who use CO2 lasers to cut the vaginal vault, while a plastic tube is used by those who use electrocautery, as it does not conduct electricity.

Currently, the surgeon requires a pelvic assistant, usually a nurse or a doctor, during laparoscopic surgery. Fatigue and musculo-skeletal problems are some of the costs to the pelvic assistant. For the hospital, it is costly to assign staff to carry out mechanical and time-consuming tasks. The assistant will be required from time to time, at the direction of the surgeon, to change the position of the manipulator or vaginal tube. Firm pressure is required to keep the vaginal vault taut and while it is being divided from the cervix. Failure to maintain pressure on the vaginal vault, via the tube, may result in excessive bleeding. The vaginal wall is stretched, resulting in the blood vessels in the vaginal tissue being compressed by the pressure of the tube against the vaginal vault, thus resulting in the blood vessels being sealed during incision of the vaginal vault. The bladder may also be in danger of being damaged if pressure is lost during excision, as the edge of the vaginal tube keeps the bladder away from the vaginal wall as it is being incised. Uncontrolled or sudden loss of pressure and the tube against the vagina may result in excessive bleeding or bladder damage.

Additionally, from time to time during the procedure an assistant is required to rotate the vaginal tube and hold it in position as requested by the surgeon.

Laparoscopic pelvic floor repair requires a vaginal probe and a rectal probe to delineate both structures to the laparoscopic surgeon. A pelvic assistant is therefore required to hold both probes.

Several prior art instruments have been specifically designed to serve the function of uterine elevation and mobilization. These include a tenaculum attached to the cervix, a metal sound inserted into the uterus, a combination instrument of a tenaculum and sound, a large curette, a modified Fletcher after-loading tandem with Teflon guard, a vacuum uterine cannula, the Semm's vacuum cannula, the Kahn balloon cannula, and the Cohen-Eder self-retaining cannula. These instruments are generally useful, but share two distinct disadvantages.

First, the force of elevation is directed against the cervix and with the exception of the metal sound and curette only splinting of the uterine cavity is provided. The above applications are potentially safe, but do not provide adequate fundal elevation, particularly in patients with soft, retroverted uteri.

Second, uterine perforation can occur if force is inadvertently directed against the uterine corpus or fundus through the proximally protruding metal rod. Effective fundal elevation can be obtained with the use of a protruding metal rod, but the application is potentially more hazardous. Uterine perforations have been reported with the use of existing instruments.

Therefore, there is a need for a uterine cannula that is safe if a force is directed against the uterine corpus or fundus, is useful to permit uterine insufflation and injection, and also can be operated to effectively seal the cervix during insufflation and injection. It is, of course, desirable for such an instrument to be relatively inexpensive, simple to manufacture and simple to operate.

In other gynecological procedures such as pelviscopy, hysteroscopy, urethroscopy and the other similar procedures, which broadly classified as minimally invasive surgical procedures, endoscopes can also be utilized. There are many disadvantages relating to current minimally invasive surgical (MIS) technology. For example, existing MIS instruments deny the surgeon the flexibility of tool placement found in open surgery. Most current laparoscopic tools have rigid shafts, so that it can be difficult to approach the worksite through the small incision. Additionally, the length and construction of many endoscopic instruments reduces the surgeon's ability to feel forces exerted by tissues and organs on the end effector of the associated tool. The lack of dexterity and sensitivity of endoscopic tools is a major impediment to the expansion of minimally invasive surgery.

Additionally, in the obstetrica vgynecological arena, for example, access into the uterus and fallopian tubes can be achieved by inserting a speculum into the vagina and performing a dilatation of the cervix if necessary or desired, such as with the use of cervical clamps. Visualization and manipulation instruments can then be inserted through the speculum opening to carry out a desired procedure. Among the devices currently used is a flexible hysteroscope, which may include visualization means such as fiber optic illumination and viewing elements and forceps-type grasping jaws.

A particular problem with the current devices and methods, however, is that multiple placements of the speculum are usually required, and the cervix is difficult to restrain in a dilated position, as it is biased to a closed position.

It would therefore be useful to develop more effective devices for performing such gynecological procedures. Specifically, it would be beneficial to develop a cannula for such uses that is flexible and expandable, thereby enabling the cannula to be utilized in a number of gynecological procedures.

In another application, traditional surgical procedures for pathologies located deep within the body, the procedure can cause significant trauma to the intervening tissues. These open procedures often require a long incision, extensive muscle stripping, and prolonged retraction of tissues, denervation and devascularization of tissue. Most of these surgeries require a recovery room time of several hours and several weeks of post-operative recovery time due to the use of general anesthesia and the destruction of tissue during the surgical procedure. In some cases, these invasive procedures lead to permanent scarring and pain that can be more severe than the pain leading to the surgical intervention.

Minimally invasive alternatives such as arthroscopic techniques reduce pain, post-operative recovery time and the destruction of healthy tissue. Orthopedic surgical patients have particularly benefited from minimally invasive surgical techniques. The site of pathology is accessed through portals rather than through a significant incision thus preserving the integrity of the intervening tissues. These minimally invasive techniques also often require only local anesthesia. The avoidance of general anesthesia reduces post-operative recovery time and the risk of complications.

Minimally invasive surgical techniques are particularly desirable for spinal and neurosurgical applications because of the need for access to locations deep within the body and the danger of damage to vital intervening tissues. For example, a common open procedure for disc herniation, laminectomy followed by discectomy requires stripping or dissection of the major muscles of the back to expose the spine. In a posterior approach, tissue including spinal nerves and blood vessels around the dural sac, ligaments and muscle must be retracted to clear a channel from the skin to the disc. These procedures normally take at least one-two hours to perform under general anesthesia and require post-operative recovery periods of at least several weeks. In addition to the long recovery time, the destruction of tissue is a major disadvantage of open spinal procedures. This aspect of open procedures is even more invasive when the discectomy is accompanied by fusion of the adjacent vertebrae. Many patients are reluctant to seek surgery as a solution to pain caused by herniated discs and other spinal conditions because of the severe pain sometimes associated with the muscle dissection.

In order to reduce the post-operative recovery time and pain associated with spinal and other procedures, micro-surgical techniques have been developed. For example, in micro-surgical discectomies, the disc is accessed by cutting a channel from the surface of the patient's back to the disc through a small incision. An operating microscope or loupes is used to visualize the surgical field. Small diameter micro-surgical instruments are passed through the small incision and between two laminae and into the disc. The intervening tissues are disrupted less because the incision is smaller. Although these micro-surgical procedures are less invasive, they still involve some of the same complications associated with open procedures, such as injury to the nerve root and dural sac, perineural scar formation, reherniation at the surgical site and instability due to excess bone removal.

Other attempts have been made for minimally invasive procedures to correct symptomatic spinal conditions. One example is chemonucleolysis that involved the injection of an enzyme into the disc to partially dissolve the nucleus to alleviate disc herniation. Unfortunately, the enzyme, chymopapain, has been plagued by concerns about both its effectiveness and complications such as severe spasms, post-operative pain and sensitivity reactions including anaphylactic shock.

Other procedures have been developed which include arthroscopic visualization of the spine and intervening structures. U.S. Pat. Nos. 4,573,448 and 5,395,317 to Kambin disclose percutaneous decompression of herniated discs with a posterolateral approach. Fragments of the herniated disc are evacuated through a cannula positioned against the annulus. The '317 Kambin patent discloses a biportal procedure which involves percutaneously placing both a working cannula and a visualization cannula for an endoscope. The procedure allows simultaneous visualization and suction, irrigation and resection in disc procedures.

Unfortunately, disadvantages remain with these procedures and the accompanying tools because they are limited to a specific application or approach. For example, Jacobson, Kambin and other references require a lateral or a posterolateral approach for percutaneous discectomy. These approaches seek to avoid damage to soft tissue structures and the need for bone removal because it was thought to be impractical to cut and remove bone through a channel. However, these approaches do not address other spinal conditions that may require a mid-line approach, removal of bone or implants.

U.S. Pat. No. 5,439,464 to Shapiro discloses a method and instruments for performing arthroscopic spinal surgeries such as laminectomies and fusions with a mid-line or medial posterior approach using three cannulas. Each of the cannulas requires a separate incision. While Shapiro discloses an improvement over prior procedures that were limited to a posterolateral or lateral approach for disc work, Shapiro's procedure still suffers from many of the disadvantages of known prior percutaneous spinal surgery techniques and tools. One disadvantage of the Shapiro procedure is its requirement of a fluid working space. Another significant detriment is that the procedure requires multiple portals into the patient.

A significant need is present for techniques and instruments that permit surgical procedures in the working space under direct vision. Procedures that reduce the number of entries into the patient are also highly desirable. The fields of spinal and neuro surgery have particularly sought devices and techniques that minimize the invasion into the patient and that are streamlined and concise in their application.

### SUMMARY OF THE INVENTION

According to the present invention, there is provided a floating surgical cannula. A method of forming a surgical cannula by inserting a floating surgical cannula at a location in need of surgery is provided.

### DESCRIPTION OF THE DRAWINGS

Other advantages of the present invention are readily appreciated as the same becomes better understood by reference to the following detailed description, when considered in connection with the accompanying drawings wherein:

FIG. 1 is a side view of the cannula of the present invention; and

FIG. 2 is a side view of the cannula of the present invention, wherein an endoscope is attached to the cannula.

### DETAILED DESCRIPTION OF THE PREFERRED EMBODIMENTS

Generally, the present invention provides a device and method for the retraction of tissues in order to form and retain a cannula for performing surgery. The cannula of the present invention can be used for the insertion of surgical instruments. The cannula of the present invention provides optimal illumination and enhanced visualization. Further, the cannula of the present invention enables surgery to be performed in a minimally invasive manner, in order to make possible a shorter recovery period for the patient.

A “cannula” refers to a surgical tube inserted into a body cavity, duct, or tissue to drain fluid, deliver medication, or allow surgery to be performed at a remote site by inserting instruments through the cannula. A cannula in this application is alternatively called a “corridor,” and can be referred to by others by various names. The cannula can be of a variety of sizes, the size of which depend upon the use of the cannula. In other words, the cannula can be larger depending upon the specific use of the cannula.

The term “expandable” as used herein is intended to refer to the ability of the material that forms the cannula to increase in width. Such expansion is controllable based upon the materials used to formulate the cannula. Alternatively, the expansion, and subsequent contraction, can occur utilizing an instrument that is capable of both expanding and contracting the cannula. Examples of such instruments are known to those of skill in the art.

The term “float” as used herein is intended to connote that the cannula is not rigidly affixed to the body at the location of insertion. The fact that the cannula “floats” enables the surgeon to manipulate the angle of entry of the instruments into the cannula and accordingly the angle at which surgery occurs.

In general, the present invention provides a device for the retraction of tissues and insertion of instruments. The device is an expandable, disposable cannula having a tubular shape. The cannula is a hollow tube constructed of a material that is expandable and retains some memory, thus enabling the cannula to always return to a pre-determined diameter after being coiled up to a smaller size. An example of such a material includes, but is not limited to, a cellulose acetate material or shape memory polymers and metals, such as nitinol.

In general, a shape memory material undergoes a change of crystal structure at its transformation temperature. Superelasticity, or pseudo elasticity, occurs when a material is in an environment that is above the temperature of its transformation temperature. The lower temperature crystal structure can be formed by applying stress to the material. Once sufficient stress is applied to the material above the transformation stress, the material undergoes deformation. Upon releasing the applied stress, the material returns to its original shape with no permanent deformation.

Any other material exhibiting shape memory behavior can also be used. For example, thermoplastic polymers can be used. A thermoplastic polymer can have one shape at room temperature, and transform into another shape at body temperature. The cannula can also be made from other materials, such as a flexible plastic. When the cannula is not made from a material exhibiting shape memory behavior, expansion can be accomplished in other ways.

Shape memory polymers can be thermoplastic, thermoset, interpenetrating networks, semi-interpenetrating networks, or mixed networks. Polymers can be a single polymer or a blend of polymers. Polymers can be linear, branched, thermoplastic elastomers with side chains or any kind of dendritic structural elements. Stimuli causing shape change can be temperature, ionic change, pH, light, electric field, magnetic field or ultrasound. The polymers can include metallic alloys.

Thermoplastic shape memory materials can be shaped (e.g. molded) to a desired shape above the Tₜᵣₐₙₛ of the hard segment(s) and cooled to a temperature below the shape recovering temperature, where the polymer can undergo mechanical deformation, and strains are generated in the polymer. The original shape of the deformed polymers can be recovered by heating them to a temperature higher than their shape recovering temperature. Above this temperature, the strains in the polymer are relieved, allowing the polymer to return to its original shape. In contrast, thermoset shape memory materials are shaped to a desired shape before the macromonomers used to form the thermoset polymers are polymerized. After the shape has been fixed, the macromonomers then are polymerized.

The polymer compositions are preferably compressible by at least one percent or expandable by at least five percent of the original thickness at a temperature below the shape recovering temperature, with the deformation being fixed by application of a stimulus such as heat, light, ultrasound, magnetic fields or electric fields.

When significant stress is applied, resulting in an enforced mechanical deformation at a temperature lower than the shape recovering temperature, strains are retained in the soft segments, or amorphous regions, and bulky shape change is kept even after the partial liberation of strain by the elasticity of the polymer. If the configuration of the molecular chains is disturbed by influencing the regulated arrangement of molecular chains at a temperature lower than the glass transition temperature, rearrangement of the molecular chains is assumed to occur through the increase of the volume size and the decrease of the free volume content. The original shape is recovered by the contraction of the hard segment aggregates by the elevation of the temperature according to rigid control of chain conformations and the shape of the polymer is restored to the memorized shape.

In addition to changes in state from a solid to liquid state (melting point or glass transition temperature), hard or soft segments can undergo ionic interactions involving polyelectrolyte segments or supramolecular effects based on highly organized hydrogen bonds. The SMP can undergo solid state to solid-state transitions (e.g. a change in morphology). Solid state to solid state transitions are well known to those of skill in the art, for example as in poly(styrene-block-butadiene).

An object formed using shape memory polymers can be prepared to control the direction of change during recovery. In other words, contraction and/or expansion can occur along one or more dimensional axes depending how the polymers are shaped and stressed. For example, in a SMP fiber, the change in shape can be limited to one dimension, such as along the length.

In another embodiment, the thermal and electrical conductivity of the SMP materials can be changed in response to changes in temperature.

The moisture permeability of the compositions can be varied, especially when the polymer is formed into a thin film (i.e., less than about 10 μm). Some polymer compositions, in their original shape, have a sufficient permeability such that molecules of water vapor can be transmitted through the polymer film, while water molecules are not large enough to penetrate the polymer film. The resulting materials have low moisture permeability at temperatures below room temperature and high moisture permeability at temperatures above room temperature.

The transition temperature at which the polymer abruptly becomes soft and deforms can be controlled by changing the monomer composition and the kind of monomer, which enables one to adjust the shape memory effect to give a desired recovery temperature. The thermal properties of the polymers can be detected, for example, by dynamic mechanical thermoanalysis (DMTA) or differential scanning calorimetry (DSC) studies. In addition the melting point can be determined using a standard melting point apparatus.

Biologicals or chemicals can be incorporated on the surface of shape memory polymer cannula that can be released or directly interact with surrounding tissue to modify tissue reactivity and promote or inhibit cell and extracellular matrix adhesion. Examples of such materials include, but are not limited to, immunosuppressive compounds and agents. Immunosuppressive agents are defined as agents that suppress immune responses. The agents can include, but are not limited to, immunoprotective cells, such as Sertoli cells, stem cells, stem cell by-products, or other compounds that create an immunosuppressive effect. Examples of such immunosuppressive compounds include, but are not limited to, TOR inhibitors, corticosteroids, cyclosporins, ascomycins, antimetabolites, alkylating agents, folic-acid antagonists, PKC inhibitors, and glutamate receptor inhibitors. A glutamate receptor inhibitor is defined as any of a class of pharmacological agents that prevent the binding and/or action of glutamate (or glutamatergic agonists) at ionotropic or metabotropic glutamate receptors, resulting in reduced or completely blocked transduction by such receptors.

The present invention is directed to a cannula for receiving surgical instruments for performing a surgical procedure on the body of a patient. The present invention is applicable to a variety of surgical procedures in which endoscopic surgical techniques are used. Surgery can be performed through the established cannula. For example, an adaptor can be included that can hold the cannula in place. Additionally, a catheter can be used to insert the cannula in place for the surgery.

More specifically, the surgical cannula of the present invention provides a minimally invasive, expandable, disposable cannula sized such that the cannula expands from, for example, 3-4 mm to 20 mm, which when combined with stereotactic guidance and the operating microscope, provides comfortable access to most deep-seated tumors or other objects that are difficult to access. The cannula is adjustable to enable the cannula to fit into various sized openings. The cannula, when coiled up, is inserted into the body at the location of a small opening that extends to the lesion/location of interest. The cannula is then allowed to open/expand, thereby providing the surgeon with sufficient surgical exposure. The exposure gained provides adequate room for excellent illumination, use of the bipolar electrocautery, use of the ultrasonic aspirator, or use of any other equipment necessary for the specific procedure.

The device of the present invention differs from the previously used rigid systems in that it is attached only to the surrounding tissue and “floats” with the tissue. The surgeon can gently manipulate the cannula by tilting it in any direction to gain additional exposure of the underlying structure. The cannula is preferably tinted. In the preferred embodiment the tint is dark-gray to avoid any light reflection from the microscope light, which can interfere with visualization. However, other colors can be used as long as the colors do not interfere with visualization.

In contrast to current endoscopic surgical techniques, the present invention has the advantage of utilizing conventional instruments, e.g. to maximize dissection and tumor removal. The present invention also allows bleeding to be controlled using conventional means. Furthermore, the present invention allows a scope to be mounted, so that both hands of a surgeon are free to manipulate instruments.

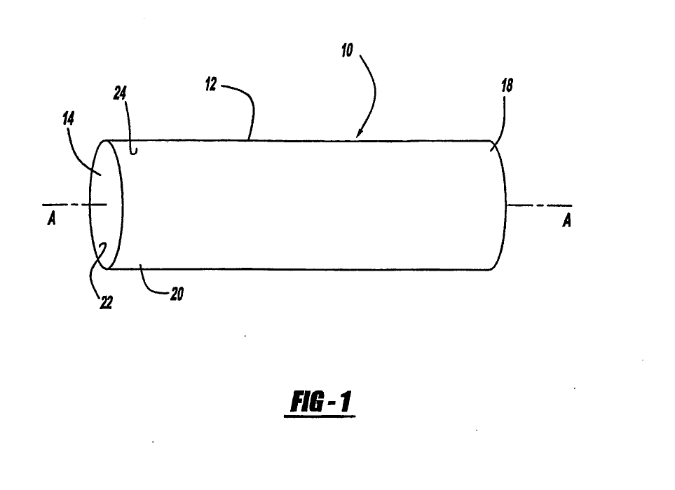

More specifically, FIG. 1 depicts a cannula 10 constructed according to the present invention. The cannula 10 is a tubular structure 12 centered on an axis A. The tubular structure defines a passage 14 through the cannula 10. Surgical instruments 16 are inserted into the body during endoscopic surgery through the passage 14.

The tubular structure 12 comprises a first tubular portion 18 and a second tubular portion 20 attached to the first tubular portion 18. The first tubular portion 18 is preferably made of a length of stainless steel tubing, but could alternatively be made of another suitable material. The first tubular portion 18 has a proximal end 22 and a distal end 24. Parallel cylindrical inner 26 and outer surfaces 28, respectively, extend between the ends 22, 24 of the first tubular portion 18. The inner surface 26 defines a first passage portion 30 of the passage 14 through the cannula 10. The first passage portion 30 has a diameter that is preferably in the range from 10 mm to 20 mm.

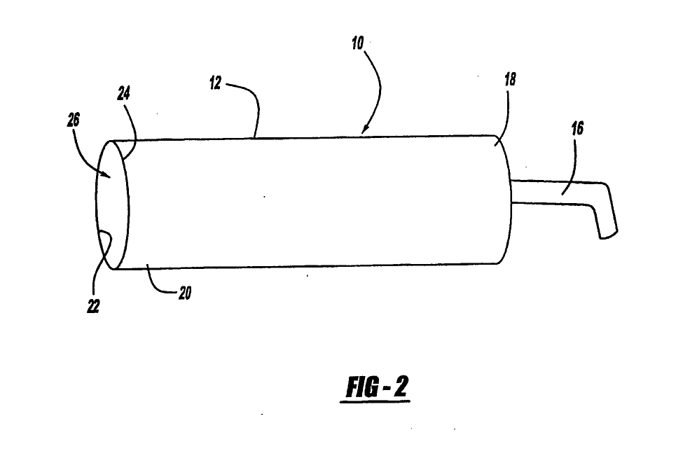

Cannula assembly 10 contemplates any configuration or apparatus allowing the optics to be supported adjacent the working channel. In the embodiment shown in FIG. 2, a fixture 16 is provided for mounting endoscope assembly on the cannula 10 with elongated viewing element disposed in working channel of cannula 10. Fixture can include a clamp attachable to the second end of cannula. Clamp is clamped on outer surface of cannula and maintains the opening for working channel at proximal end. The working channel is sized to receive one or more surgical tools therethrough for performing surgical procedures through cannula.

Cannula assembly can also include irrigation and aspiration components extending along viewing element in cannula. Endoscope assembly includes a detachable endoscope that is removable from clamp. Endoscope assemblies are well known to those of skill in the art. Cannulas and endoscope assemblies are also described in U.S. Pat. Nos. 5,792,044 and 5,902,231 to Foley et al., which patents are also incorporated herein by reference in their entirety.

The cannula of the present invention can be used for the retraction of tissues and insertion of instruments in advanced surgeries, such as microscopic or endoscopic surgery, for intracranial procedures, including supratentorial tumor resection, evacuation of spontaneous intracranial hemorrhages, ablative epilepsy surgery, treatment of intracerebral abscesses, aneurysm clipping, spinal discectomies, and gynecological procedures. Since, the cannula is a minimally invasive brain retraction system, which is extremely useful, the cannula provides an additional tool that improves the resection of intracranial tumors.

The present invention is advantageous over prior art devices for a number of reasons. For example, when used in brain surgery, the system and method of the present invention allows the surgery to be performed in the traditional fashion with conventional instruments, while utilizing a small cannula. Additionally, brain surgeries incorporating the present invention can still be performed without requiring the surgeons to undergo costly retraining.

Additionally, the present invention provides enhanced visualization, such that direct line of sight is not required, and that the surgical field is better illuminated. When used in brain surgery, the present invention allows for a smaller craniotomy, as well as less brain tissue retraction. Furthermore, the present invention can be used with stereotactic planning systems. Still further, the present invention is applicable in a number of types of surgeries, including intracranial neurosurgery.

Further, the cannula of the present invention can be used in a wide range of surgical procedures, and particularly spinal procedures such as cervical spine surgery, laminotomy, laminectomy, foramenotomy, facetectomy and discectomy, using a posterior, postero-lateral, or lateral approach to the disc space. The devices and instruments of present invention have application to inventive surgical techniques that permit each of these several types of surgical procedures to be performed via a single working channel. The present invention also has application to surgical techniques for preparing a disc space for insertion of an implant into the disc space. The present invention further has application in a transforaminal, minimally invasive surgical procedure in which the disc space is prepared for insertion of one or more implants into the disc space with a unilateral approach.

The present invention also contemplates instruments for use with the cannula assembly to prepare a disc space for insertion of one or more implants and inserting the implants in the disc space. Specific instruments include distractors, shims, chisels, distractor-cutters, implant holders, reamers, and drills. Other instruments for performing surgical procedures on the vertebral bodies or in the disc space are also contemplated herein as would occur to those skilled in the art so long as the instruments are capable of being used in a minimally invasive procedure through working channel of cannula.

The cannula of the present invention can also be used in a wide range of gynecological procedures, and particularly surgical procedures such as hysteroscopy. The devices and instruments of present invention have application to inventive surgical techniques that permit each of these several types of surgical procedures to be performed via a single working channel. The present invention also has application to surgical techniques for non-surgical procedures requiring the cervix or uterus to be accessed. For example, the cannula can be used for dilatation and curettage, both diagnostic and non-diagnostic, surgical abortion, and other similar surgical and non-surgical gynecological procedures.

The method and apparatus of the present invention are exemplified in the description. While specific embodiments are disclosed herein, they are not exhaustive and can include other suitable designs that vary in design and methodologies known to those of skill in the art. Basically, any differing design, methods, structures, and materials known to those skilled in the art can be utilized without departing from the spirit of the present invention.

Throughout this application, author and year, and patents, by number, reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

What is claimed is:

1. A floating surgical cannula.

2. The surgical cannula according to claim 1, wherein said surgical cannula is expandable.

3. The surgical cannula according to claim 2, wherein said surgical cannula can expand to 20mm.

4. The surgical cannula according to claim 1, wherein said surgical cannula is disposable.

5. The surgical cannula according to claim 1, wherein said surgical cannula is made of expandable material having memory.

6. The surgical cannula according to claim 4, wherein said material is cellulose acetate material.

7. The surgical cannula according to claim 5, wherein said material is a shape memory polymer.

8. The surgical cannula according to claim 7, wherein said shape memory alloy is nitinol.

9. The surgical cannula according to claim 1, further including surgical tools.

10. The surgical cannula according to claim 9, wherein said surgical tools are selected from the group consisting essentially of an aspirator, an irrigator, distractors, shims, chisels, disctractor-cutters, implant holder, reamers, drills, cureftes, endoscopes, and other visualizing means.

11. The surgical cannula according to claim 1, further including a coating on an exterior surface of said cannula.

12. The surgical cannula according to claim 11, wherein said coating is an coating capable of modifying tissue reactivity.

13. The surgical cannula according to claim 12, wherein said coating is an immunosuppressive coating.

14. The surgical cannula according to claim 13, wherein said immunosuppressive coating is selected from the group consisting essentially of immunoprotective cells, stem cells, stem cell by-products, TOR inhibitors, corticosteroids, cyclosporins, ascomycins, antimetabolites, alkylating agents, folic-acid antagonists, PKC inhibitors, and glutamate receptor inhibitors.

15. A method of forming a surgical cannula by inserting a floating surgical cannula at a location in need of surgery.

16. The method according to claim 15, wherein said inserting step includes forming a small opening at the location in need of surgery, inserting a contracted floating surgical cannula at the location, and expanding the surgical cannula to a size sufficient for surgery.

17. The method according to claim 15, wherein said inserting step includes expanding the surgical cannula using a heating mechanism.

18. A device for use in surgical and non-surgical procedures, said device comprising a floating cannula.

19. The device according to claim 18, wherein said surgical cannula is expandable.

20. The device according to claim 19, wherein said surgical cannula can expand to 20mm.

21. The device according to claim 18, wherein said surgical cannula is disposable.

22. The device according to claim 18, wherein said surgical cannula is made of expandable material having memory.

23. The device according to claim 22, wherein said material is cellulose acetate material.

24. The device according to claim 22, wherein said material is a shape memory polymer.

25. The device according to claim 24, wherein said shape memory alloy is nitinol.

26. The device according to claim 18, further including surgical tools.

27. The device according to claim 18, wherein said device can be used for procedures selected from the group consisting essentially of neurological procedures, spinal procedures, and gynecological procedures.

28. The device according to claim 27, wherein said neurological procedure is selected from the group consisting essentially of intracranial procedures, supratentorial tumor resection, evacuation of spontaneous intracranial hemorrhages, ablative epilepsy surgery, treatment of intracerebral abscesses, and aneurysm clipping.

29. The device according to claim 27, wherein said spinal procedure is selected from the group consisting essentially of laminotomy, laminectomy, foramenotomy, facetectomy and discectomy, using posterior, postero-lateral, and lateral approach to the disc space.

30. The device according to claim 27, wherein said gynecological procedure is selected from the group consisting essentially of laparoscopy, hysteroscopy, dilatation and curettage, and non-surgical gynecological procedures.
